(19) United States
(12) Patent Application Publication
(10) Pub. No.: US 2014/0012264 A1
(43) Pub. Date: Jan. 9, 2014

(54) TOTAL HIP ARTHROPLASTY

(71) Applicant: TOTAL JOINT ORTHOPEDICS, Salt Lake City, UT (US)

(72) Inventors: Robert G. Prybyla, Round Rock, TX (US); Benjamin C. Casey, Salt Lake City, UT (US)

(73) Assignee: TOTAL JOINT ORTHOPEDICS, Salt Lake City, UT (US)

(21) Appl. No.: 14/024,146

(22) Filed: Sep. 11, 2013

Related U.S. Application Data
(62) Division of application No. 13/442,222, filed on Apr. 9, 2012.
(60) Provisional application No. 61/474,910, filed on Apr. 13, 2011.

(51) Int. Cl. A61B 17/16

(52) U.S. Cl. CPC A61B 17/1668
USPC 606/80; 606/79

(57) ABSTRACT

A total hip arthroplasty includes a femoral prosthesis and an acetabular prosthesis. The femoral prosthesis has a ball on a neck portion of a femoral component including a noncircular femoral body a portion coated with a porous bone ingrowth coating for noncemented fixation within a resected patient femur. The femoral body is tapered, to include a combination of a medial-lateral taper, an anterior-posterior taper, and a lateral-to-medial taper for secure seated fixation into the medullary canal of a resected femur. The acetabular prosthesis includes a hemispherical metal cup having a porous bone ingrowth surface for secure fixation within a prepared patient acetabular socket. A bearing insert is initially seated within the acetabular cup in a “trial” position without locking to determine cup placement and freedom of leg movement, and thereafter reseated in a “lock” position. A femoral broach and a bone punch tool are also provided.

### BACKGROUND OF THE INVENTION

This invention relates generally to an improved total hip arthroplasty, including a femoral prosthesis and an acetabular prosthesis. More particularly, this invention relates to an improved femoral prosthesis having a noncircular femoral stem or body having a combination of tapers for secure and noncemented seating within a resected patient femur. This improved femoral prosthesis is desirably used with an improved acetabular prosthesis including an acetabular cup designed for secure mounting into a prepared patient acetabular socket, and further including a bearing insert adapted for initial seating within the acetabular cup in a trial or test position to determine proper and desired cup positioning and patient leg movement, and later adapted for repositioning within the cup in a secure and stable lock position.

### SUMMARY OF THE INVENTION

In accordance with the invention, a total hip arthroplasty includes a femoral prosthesis and an acetabular prosthesis. The femoral prosthesis is defined by a ball-shaped femoral head mounted onto a neck portion of a femoral component which includes a noncircular and blade-shaped femoral body having a portion of the exterior surface thereof coated with a porous bone ingrowth coating for noncemented fixation within a resected patient femur.

In the preferred form, the noncircular femoral body is constructed from a sturdy metal or metal alloy material and includes the porous bone ingrowth coating over an upper or proximal region thereof. A noncircular femoral stem forming a portion of the femoral body protrudes downwardly or distally from this porous bone ingrowth coating, wherein this femoral stem includes a central region that is rough-textured as by grit blasting, and a lower or distal region that is smooth-surfaced for nonattachment to patient bone. The femoral body is tapered, to include a unique combination of a medial-lateral taper, an anterior-posterior taper, and a lateral-to-medial taper for secure seated fixation into the medullary canal of a resected femur.

A femoral broach of matingly tapered shape is provided for preparing the medullary canal of the resected patient femur to receive the noncircular femoral body. This femoral broach beneficially defines a series of cutting surfaces or teeth on a femoral body thereof for cutting and shaping the interior of the medullary canal to receive securely the tapered noncircular femoral component.

The acetabular prosthesis comprises includes a hemispherical cup formed from a sturdy material such as metal or metal alloy and having a porous bone ingrowth surface on a convex side thereof for secure fixation to patient bone within a prepared patient acetabular socket. A bearing insert formed preferably from a plastic material is initially seated within the acetabular cup in a “trial” position without locking to determine cup placement and freedom of leg movement. Thereafter, the bearing insert is reseated within the acetabular cup in a “lock” position with the bearing insert snap-fit attached to the acetabular cup.

A bone awl or punch tool is provided to form one or more pilot openings or holes in patient bone such as within the prepared acetabular socket each to receive a preferably self-tapping bone screw used for securing the acetabular cup to the prepared patient bone. The bone awl includes a pointed tool tip for punching a hole of predetermined size into patient bone, in combination with an enlarged shoulder at the base of said tip to limit the depth of the punched hole.

Other features and advantages of the invention will become more apparent from the following detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is an exploded perspective view of a prosthetic total hip joint constructed in accordance with the novel features of the invention, and including a femoral prosthesis and an acetabular prosthesis;

FIG. 2 is an anterior elevation view of a tapered femoral component including a noncircular femoral body and a femoral neck, and showing a medial-lateral taper for the femoral body;

FIG. 3 is medial elevation view of the femoral component of FIG. 2, and illustrating an anterior-posterior taper for the femoral body;

FIG. 4 is a sectional view taken generally on the line 4-4 of FIG. 2, and depicting a lateral-to-medial taper for the femoral body;

FIG. 5 is a fragmented perspective view showing initial gauge measurement of the neck region of an unresected patient femur;

FIG. 6 is another fragmented perspective view showing a standard bone chisel and mallet for initially preparing a resected patient femur for receiving the femoral component of the present invention;

FIG. 7 is a fragmented perspective view similar to FIG. 6, but depicting a standard reaming tool for preparing the resected patient femur;

FIG. 8 is an anterior elevation view similar to FIG. 2, but depicting a femoral broach for continued preparation of the resected patient femur;

FIG. 9 is an anterior and fragmented elevation view illustrating initial and relatively loose placement of the femoral broach into the resected femur;

FIG. 10 is a somewhat reduced size anterior elevation view similar to a portion of FIG. 7, but further illustrating a broach holder tool carrying the femoral broach for use in initial broach placement into the resected femur, and for subsequent use in retracting the broach from the resected femur;

FIG. 11 is an anterior and fragmented elevation view similar to FIG. 9, but showing final seated placement of the femoral broach into the medullary canal of the resected patient femur;

FIG. 12 is an anterior and fragmented elevation view similar to FIGS. 9 and 11, but further illustrating mounting of a trial ball onto the neck of the seated femoral broach;

FIG. 13 is an anterior and fragmented elevation view showing placement of the femoral component of FIGS. 1 and 2 into the medullary canal of a resected patient femur using a standard orthopedic stem driver and mallet;

FIG. 14 is an anterior and fragmented elevation view similar to FIG. 13, further illustrating a rod for use in further seating of the femoral component into the resected patient femur;

FIG. 15 is an anterior and fragmented elevation view showing use of a femoral head impactor for mounting the assembled femoral head with a press-fit relation onto the neck of the femoral component;

FIG. 16 is a plan view showing an open end of an acetabular cup forming a portion of the acetabular prosthesis;

FIG. 17 is a perspective view depicting a porous bone ingrowth surface applied onto the outer or convex side of the acetabular cup of FIG. 16;

FIG. 18 is a fragmented perspective view illustrating preparation of the acetabular hip socket with a conventional reaming tool;

FIG. 19 is a fragmented perspective view similar to FIG. 18, but showing use of a cup inserter rod for initially placing the acetabular cup into the prepared hip socket;

FIG. 20 is a fragmented perspective view similar to FIGS. 18-19, but showing use of the bone punch tool of FIG. 20 in preparing patient bone to receive a bone screw;

FIG. 21 is a plan view of a preferred bone punch tool for use in the invention;

FIG. 22 is a fragmented perspective view similar to FIG. 21, but illustrating use of a conventional surgical rotary drive to install at least one bone screw through the acetabular cup into patient bone;

FIG. 23 is an enlarged perspective view showing a plastic bearing insert or bushing seated within the acetabular cup in a trial position;

FIG. 24 is a plan view showing the plastic bearing insert seated within the acetabular cup in the trial position;

FIG. 25 is an enlarged and fragmented vertical sectional view taken generally on the line 25-25 of FIG. 24;

FIG. 26 is a further enlarged vertical sectional view taken generally with the encircled region 26 of FIG. 25 in the trial position;

FIG. 27 is an enlarged perspective view showing the plastic bearing insert seated within the acetabular cup, similar to FIG. 23, but showing the bearing insert in a lock position;

FIG. 28 is a plan view similar to FIG. 24 but showing the plastic bearing insert seated within the acetabular cup in the lock position;

FIG. 29 is an enlarged and fragmented vertical sectional view taken generally on the line 29-29 of FIG. 28, and showing the bearing insert in a pre-engaged lock position;

FIG. 30 is a further enlarged vertical sectional view corresponding generally with the encircled region 30 of FIG. 29;

FIG. 31 is an enlarged vertical sectional view similar to FIG. 29, but illustrating the plastic bearing insert impacted to a fully locked position within the acetabular cup;

FIG. 32 is a further enlarged and fragmented vertical sectional view corresponding generally with the encircled region 32 of FIG. 31; and

FIG. 33 is a fragmented perspective view showing use of an impactor tool for seating the plastic bearing insert in the locked position within the acetabular cup.

### DETAILED DESCRIPTION OF THE PREFERRED EMBODIMENTS

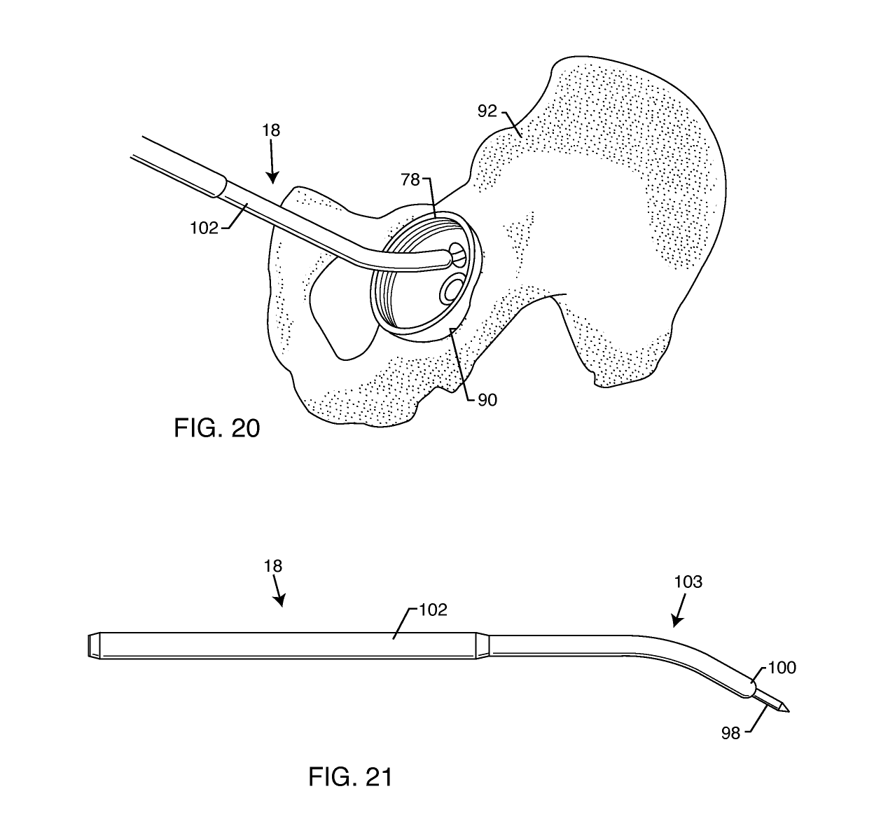

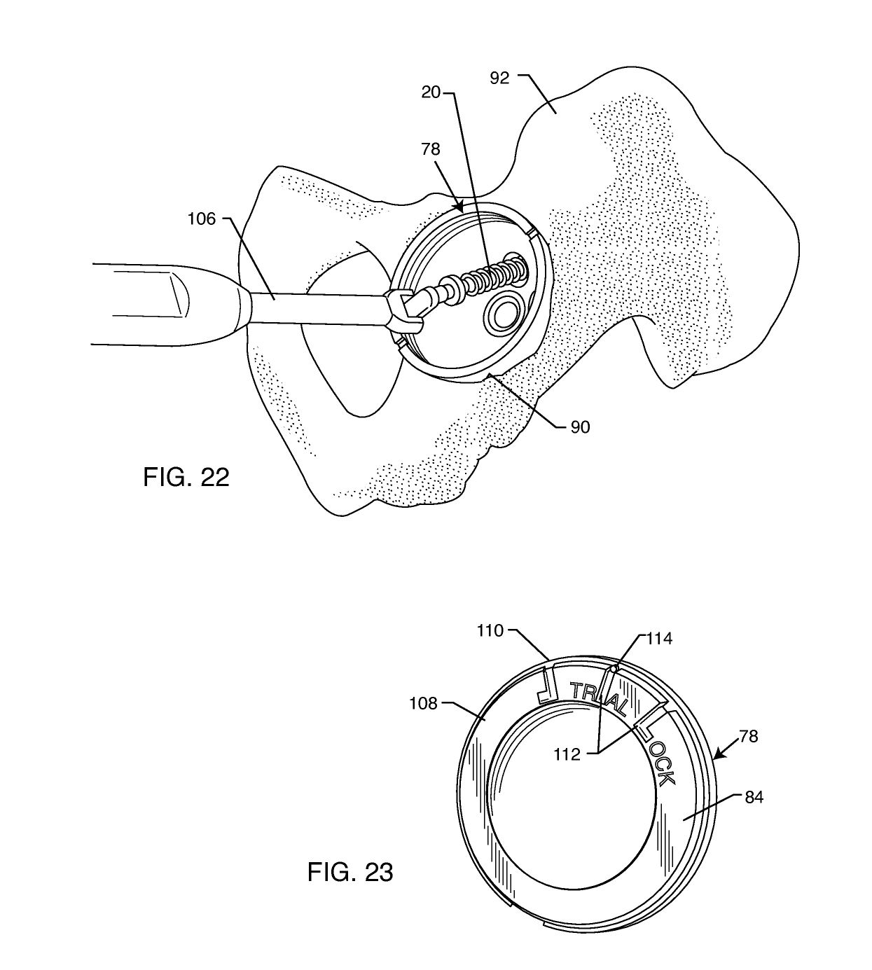

As shown in the exemplary drawings, an improved total hip arthroplasty referred to generally in FIG. 1 by the reference numeral 10 is provided to implantation into a patient (not shown in FIG. 1). The hip arthroplasty or total hip prosthesisl 10 comprises a femoral component or prosthesis 12 and a related acetabular component or prosthesis 14. The femoral prosthesis 12 is designed for improved and stable implantation into a resected femur (not shown in FIG. 1) of a patient, whereas the acetabular prosthesis 14 is designed for secure and stable implantation into a prepared acetabular socket (also not shown in FIG. 1) of a patient. A unitary or one-piece broach tool 16 (FIGS. 7-11) is also provided for use in preparing and shaping the medullary canal of the resected patient femur, and an improved bone awl or punch 18 (FIGS. 20-21) is provided for quick and easy formation of a pilot hole or opening to receive a self-tapping bone screw 20 (FIG. 22).

The femoral prosthesis 12 of the invention generally comprises a femoral component 22 having an elongated and noncircular femoral stem or body 24, in combination with an upper or proximal end including a neck portion 26 adapted for seated reception with a press-fit relation into a counterbore 28 formed in a femoral ball 30. The femoral body 24 is shown with a preferred, generally rectangular cross sectional shape extending from a relatively wide upper or proximal region downwardly or distally to a tip end 32. As shown, this femoral body 24 desirably includes an upper or proximal region carrying a porous bone ingrowth coating 34 of a type known in the art for noncemented attachment and fixation of the femoral body 24 within the medullary canal of a resected patient femur. In this regard, the femoral body is preferably constructed from a sturdy and biocompatible material such as a rigid titanium or titanium alloy or cobalt chrome alloy or the like, and the porous bone ingrowth coating 34 is formed as by a titanium sintering process of the like for metaphyseal fit and biological fixation. One preferred porous bone ingrowth coating 34 comprises Ti-Coat—a three dimensional commercially pure coating process with a mean porosity of about 61%.

Beneath or distally relative to the porous bone ingrowth coating 34, the femoral body 24 includes a surface-roughened central region 36 formed as by grit-blasting or the like during manufacture, preferably to define a surface roughness of about 3-5 microns. This surface-roughened central region 36 is designed for improved diaphyseal fixation. The central region 36 merges with the lower or distal tip end 32 which is smooth-surfaced to avoid significant bone attachment thereto in a manner to reduce stress shielding and cantilevering.

The femoral component 22 including the femoral body 24 is shown in more detail in FIGS. 2-4. As shown, the femoral body 24 is formed with a continuous medial-lateral taper (FIG. 2) which progressively increases from the distal tip end 32 through the central region 36 and continuing to at least a portion of the upper or proximal region including the porous bone ingrowth coating 34. In a preferred form, this medial-lateral taper is about 5 degrees.

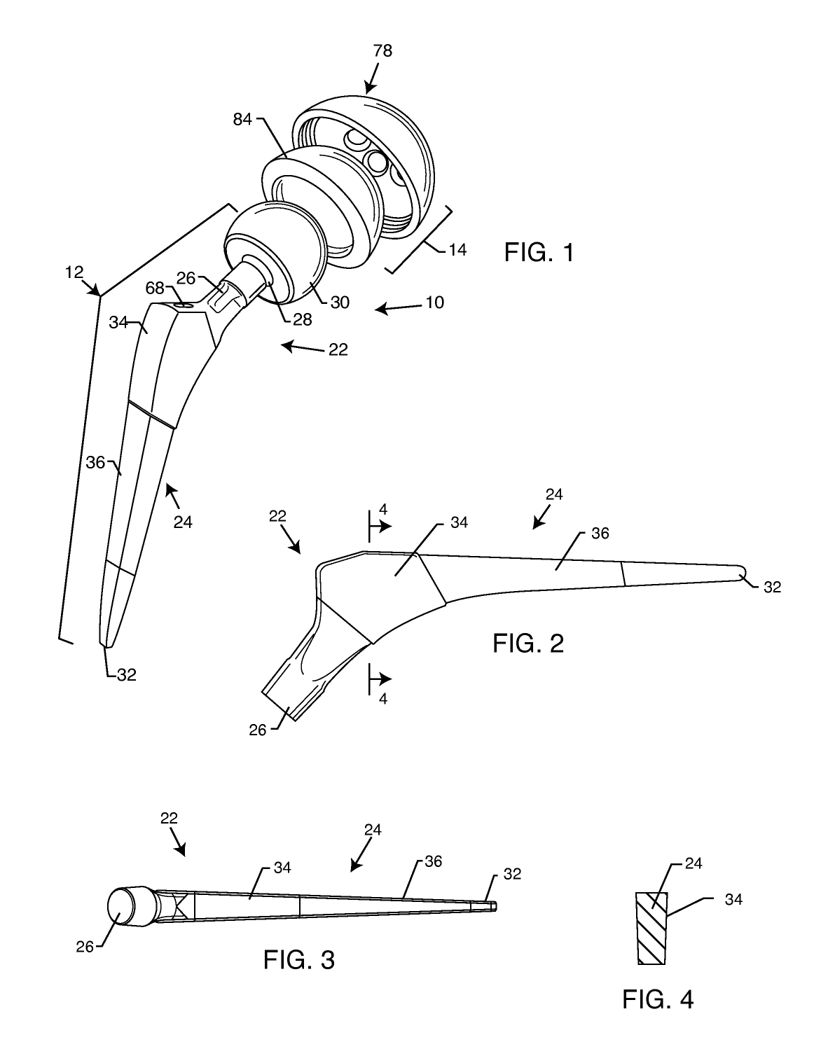

The femoral body 24 also includes an anterior-posterior taper which progressively increases substantially continuously from the distal tip end 32 through the central region 36 and the upper or proximal region including the porous bone ingrowth coating 34, as viewed in FIG. 3. In a preferred form, the anterior-posterior taper is about 3-4 degrees, and more preferably about 3.5 degrees.

The femoral body 24 further includes a lateral-to-medial taper (shown in FIG. 4) which progressively decreases substantially continuously from the lateral side to the medial side of the femoral body 24. This lateral-to-medial taper is about 4-5 degrees, and more preferably about 4.4 degrees.

This combination of three different tapers on the femoral body 24 is believed to beneficially provide improved and secure or stable seating of the femoral body 24 into the medullary canal of a resected patient femur, as will be described herein in more detail. Specifically, the combination of tapers provides a secure wedging and locking effect between the femoral body 24 and the patient bone, so that safe and secure bone ingrowth is achieved post-surgically.

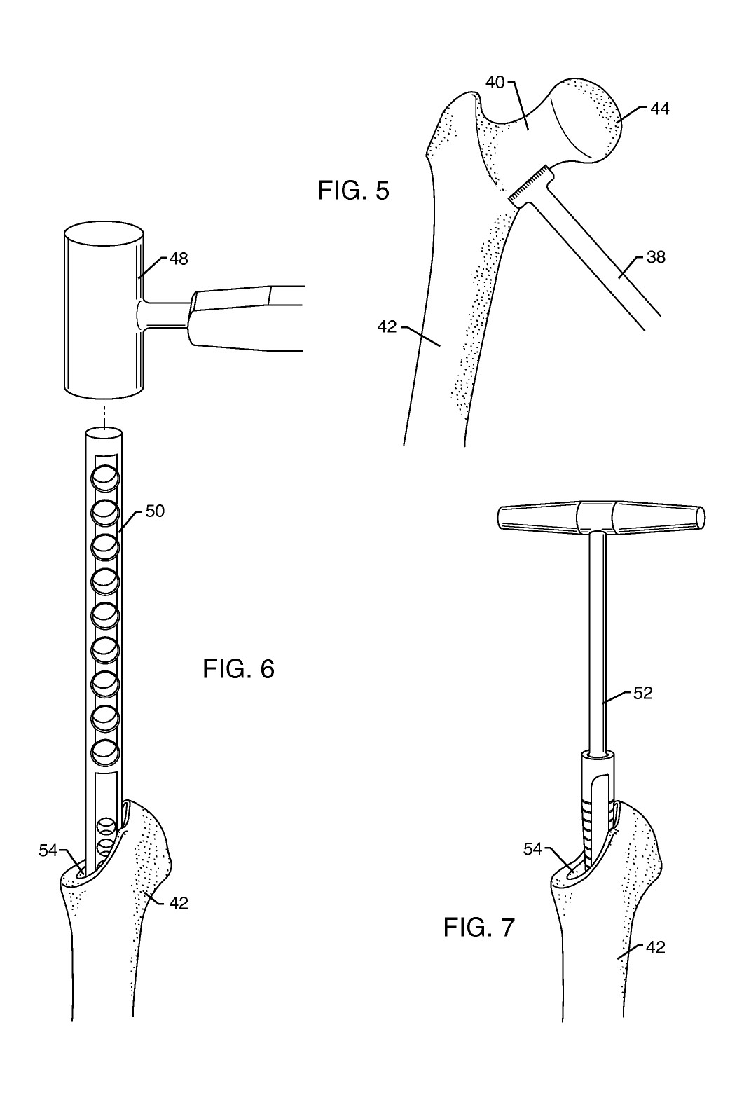

FIG. 5 shows an early step in the implantation procedure, wherein a metering device 38 is used to measure the length of the neck region 40 of a natural, unresected patient femur 42. Subsequently, a bone saw (not shown) or the like is used by the surgeon to resect or remove the natural femoral ball 44 and a portion of the neck region 26 to expose the internal medullary canal (not shown in FIG. 5) of the femur 42. In a preferred technique, the femur 42 is resected close to the base of the natural femoral neck region 40 at an angle of about 40-45 degrees, generally parallel to the intertrochanteric line. FIG. 6 shows a subsequent step including a surgical mallet 48 and a surgical box chisel 50 used to insure that sufficient cortical bone is removed and/or shaped to receive and support the femoral prosthesis 12 (FIG. 1). FIG. 7 illustrates a further surgical preparation step wherein a canal finder reaming tool 52 is employed to open up and customize the internal geometry or shape of the medullary canal 54 of the resected femur 42 to a depth corresponding with the size and shape of the femoral body 24.

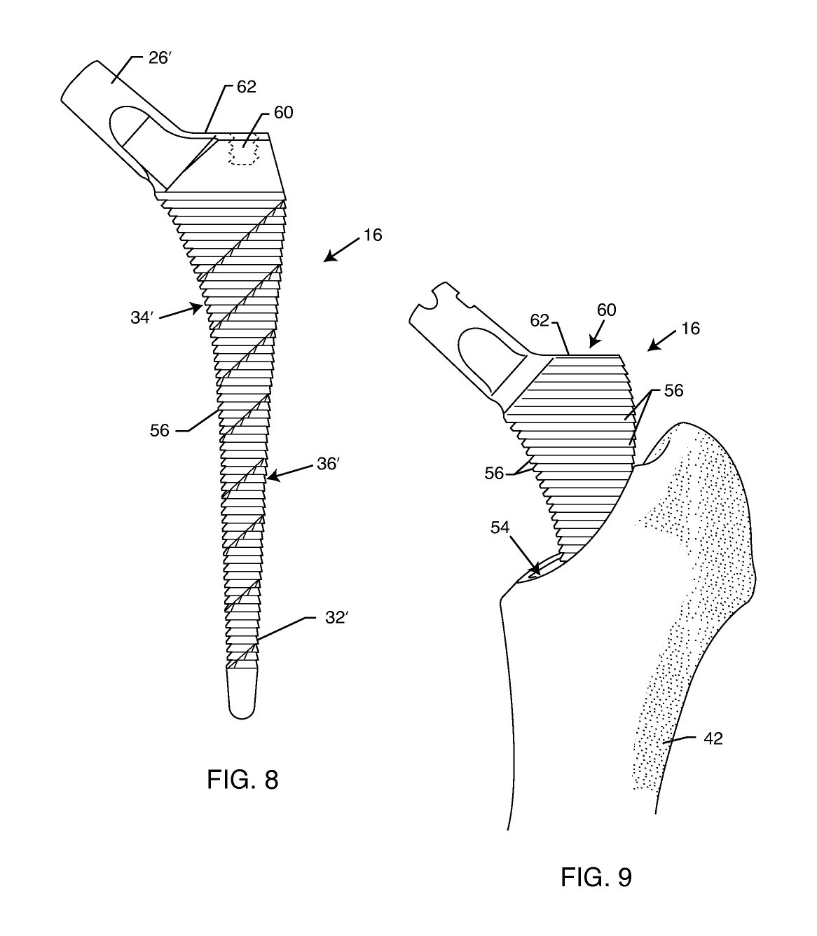

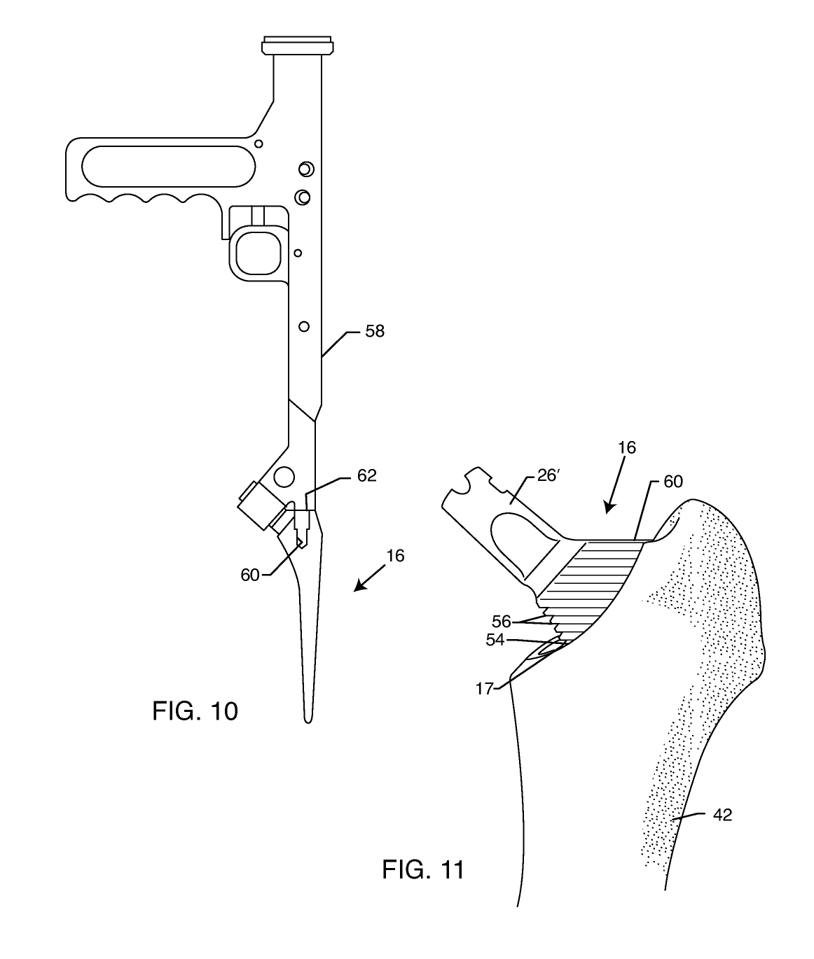

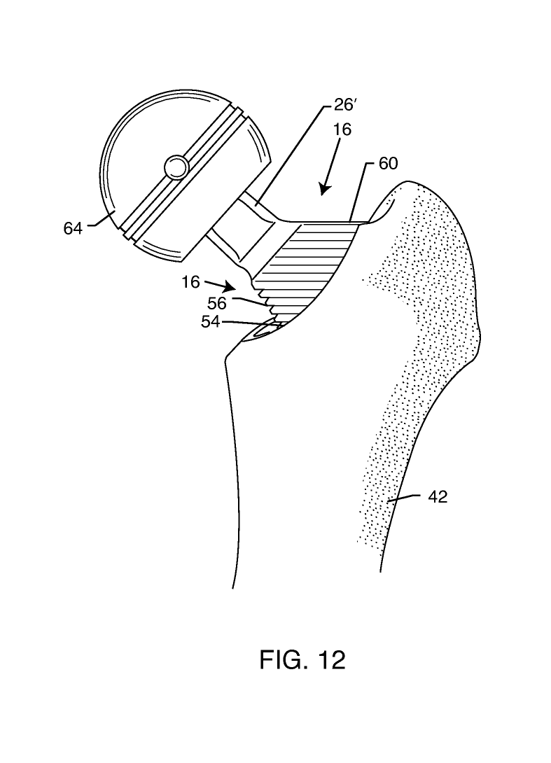

FIG. 8 shows a preferred broach or broach tool 16 for use in further shaping of the medullary canal 54 of the resected patient femur 42. In actual practice, a plurality of such broach tools 16 are used in different, progressively increasing sizes, with FIGS. 8-12 illustrating a final broach tool or femoral tool component 16 having the combination of tapers and a final size identical to the size of the femoral body 24, to include external cutting teeth 56 formed on the exterior surfaces of a distal end tip 32′, a central region 36′, and an upper or proximal end region 34′ corresponding with the porous bone ingrowth coating 34 on the femoral body 24. Each femoral broach 16 includes an etched line 17 (FIG. 11) or the like corresponding with the top surface of the porous bone ingrowth surface 34 of the femoral body 24. Each of the multiple femoral broach or broach tools 16 is preferably constructed from a suitable material such as a surgical grade and biocompatible metal or metal alloy, such as stainless steel or the like, and includes an upper neck portion 26′ suitable for grasping and retention by a broach inserter/retractor tool 58 as shown by way of example in FIG. 10. In this regard, the broach tool 16 typically additionally includes a threaded counterbore 60 or the like formed within an upper or proximal surface 62 for easy threaded coupling with the inserter/retractor tool 58. FIG. 9 shows the broach tool 16 received relatively loosely within the medullary canal 54 of the resected femur 42, whereas FIG. 11 shows the broach tool 58 fully inserted and seated within the medullary canal 54. During an initial broaching procedure, a smaller sized broach tool 16 is implanted into the medullary canal 54, and then removed therefrom so that a larger size broach tool 16 can be used. This process is repeated until the final broach 16 (viewed in FIG. 11) is fully seated within the medullary canal of the resected patient femur 42.

FIG. 12 shows a temporary ball-shaped cap 64 mounted onto the neck portion 26′ of the broach tool 16, for use in trial reduction (measuring and testing) of the actual fit between the femoral prosthesis 12 and the acetabular prosthesis 14, as will be described herein in more detail. The illustrative temporary cap 64 can be formed from any lightweight autoclavable (sterilizable) and thus reusable plastic material or the like. The process is repeated using different sized temporary caps 64 until the fit is optimized.

After suitable testing of the cap 64 for fit with the acetabular prosthesis 14, persons skilled in the art will recognize and appreciate that the temporary cap 64 can be removed quickly and easily from the broach tool 16, whereupon the broach tool 16 can be similarly quickly and easily reattached to the inserter/retractor tool 58 (FIG. 10) for pull-out removal from the resected femur 42.

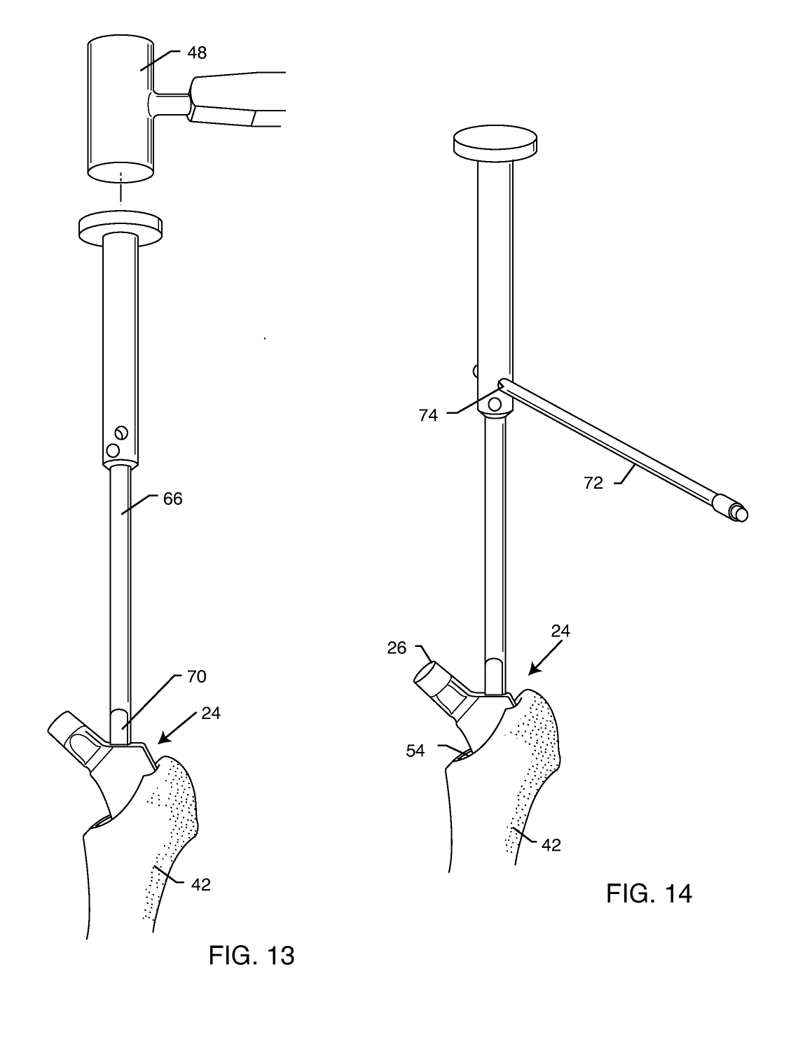

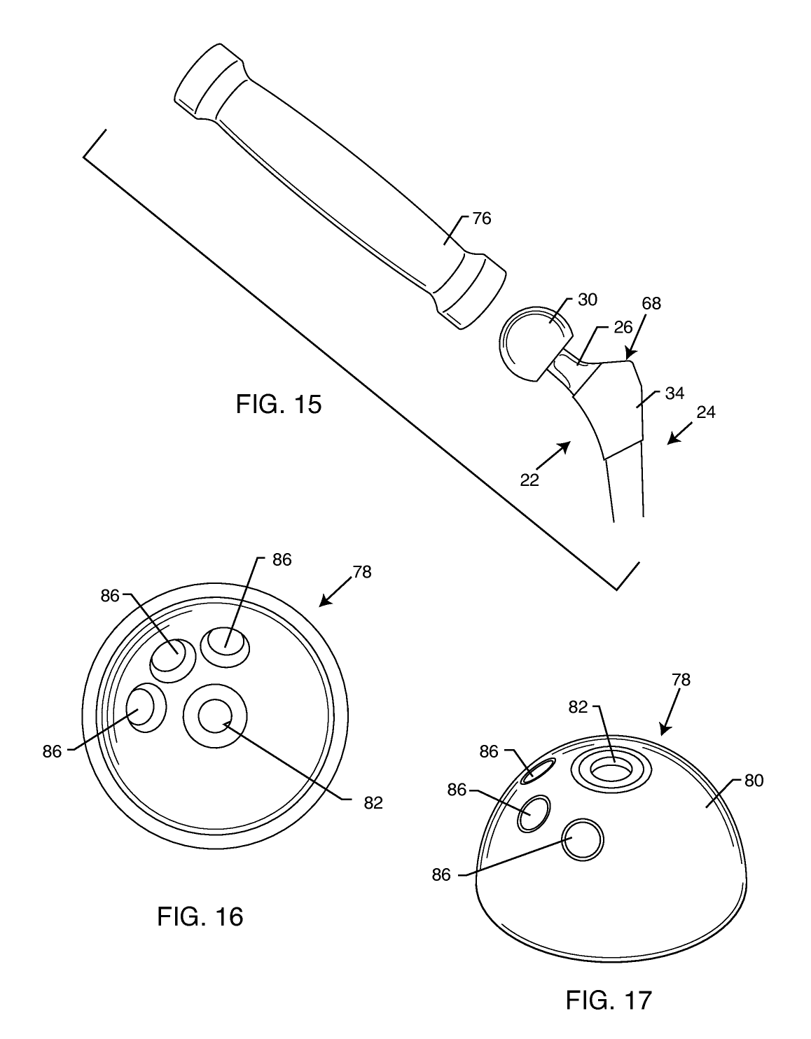

FIG. 13 shows use of the surgical hammer 48 with a chisel-like stem driver 66 for impacting the femoral body 24 into the resected femur 42. In this regard, a shallow slot 68 (shown best in FIG. 1) is formed at the top of the femoral body 24 to receive the blade-like tip 70 of the stem driver 66. An anteversion rod 72 (FIG. 14) may be fitted through a laterally open port 74 in the stem driver 66 for use in insuring proper fit of the femoral body 24 without rotation relative to the resected femur 42. After seated placement of the femoral body 24 in the final position within the medullary canal 54 of the resected femur 42, a femoral head impactor 76 (FIG. 15) is used with the mallet 48 (not shown in FIG. 15) to seat the femoral ball 30 in a tight press-fit or morse taper lock relation onto the neck portion 26 of the femoral component 22. In a preferred form, the femoral ball 30 is formed from a polished metal material such as a titanium or titanium alloy or cobalt chrome alloy to emulate the function of the natural and now removed femoral ball 44 (FIG. 5).

Persons skilled in the art will recognize and appreciate that a tapered sleeve adaptor (not shown) of selected head center offset can be used for press-fit mounting between the femoral ball 30 and the neck portion 26 of the femoral component 22, if desired or required.

In addition, use of the noncircular, multi-tapered and preferably rectangular shape of the femoral body or stem 24 in combination with the porous ingrowth coating 34 beneficially provides both secure initial and long-term fixation of the implant relative to the patient's resected femur 42. The preliminary use of the broach tool 16 beneficially prepares the patient bone and shapes the medullary canal 54 in a manner that minimally disturbs the endosteal blood supply while maximizing bone conservation.

FIGS. 16 and 17 respectively show the concave and convex sides of an acetabular cup 78 forming a portion of the acetabular prosthesis 14 (FIG. 1). As shown, the cup 78 is formed from a selected biocompatible and sturdy metal material, such as titanium or titanium alloy or cobalt chrome alloy or the like, with a generally hemispherical shape. The interior (FIG. 16) of the cup 78 is polished or smooth, whereas the exterior carries a porous bone ingrowth coating 80 similar to the porous bone ingrowth coating 34 on the femoral body 24. A central port 82 is formed in the cup 78 for secure and stable positioning of a bearing insert 84 (FIGS. 1 and 23-33) to be described herein in more detail. In addition, in the preferred form, multiple screw ports 86 are formed in the cup 78 at positions disposed off-axis and preferably in a superior direction relative to the central port 82, when the cup 78 is implanted into patient bone. In the size acetabular cup 78 as shown, three such screw ports 86 are provided.

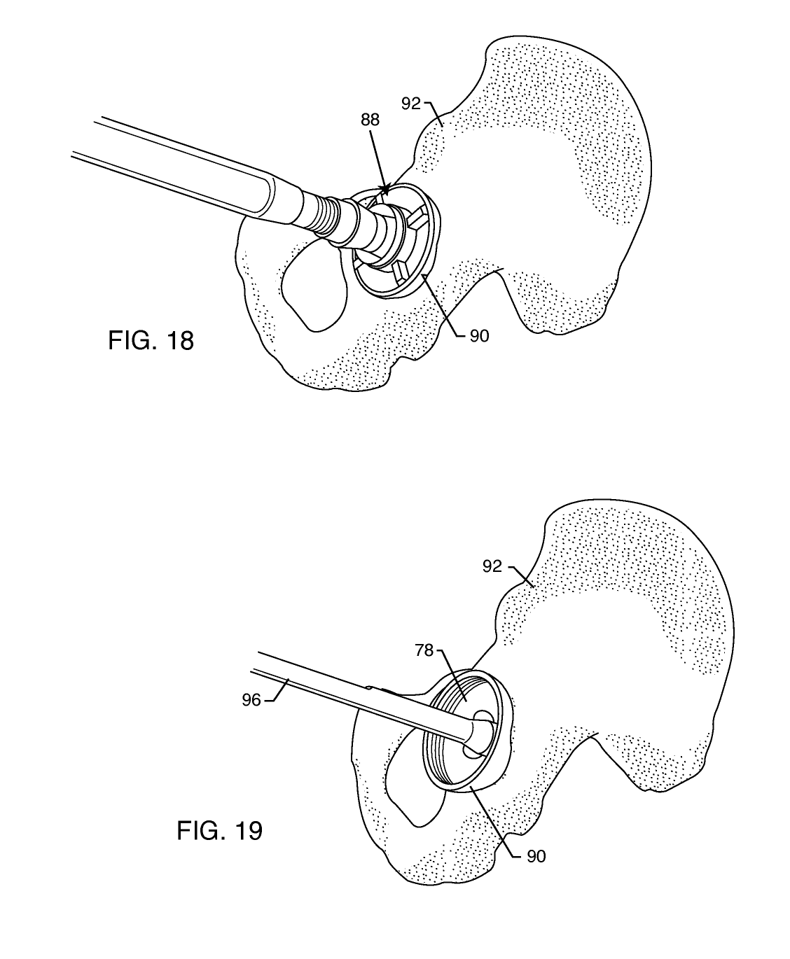

FIG. 18 depicts a standard rotary spherical reaming tool 88 used to prepare the acetabular socket 90 in patient bone 92, wherein this socket 90 forms an integral portion of the patient's natural hip joint. The reaming tool 88 is used to remove existing cartilage material and the like preparatory to implantation of the acetabular cup 78. Caution is used to avoid removal of excess patient bone 92. In practice, multiple spherical reamers 88 are used, with progressively increasing diametric size, to attain the desired diametric size of the socket 90. That is, the final reamed socket diameter should conform closely with the diametric size of the acetabular cup 78. FIG. 19 illustrates a cup inserter tool 94 having a tip end (not shown) with a threaded tip construction or the like to thread-in engagement with a mating thread 96 (FIG. 25) lining the central port 82 of the acetabular cup 78. The inserter tool 94 is used to place the cup 78 as by tapping with the mallet 48 (not shown in FIG. 19) into the prepared socket 90 until the cup exterior seats on the prepared patient bone. The specific orientation, namely, the desired abduction and anteversion of the acetabular cup 78 within the prepared socket 90, requires a degree of estimation. The inserter tool 94 is then removed.

Persons skilled in the art will appreciate that alternative forms of the inserter tool 94 may be employed, with different degrees of cup insertion accurarcy.

FIG. 20 shows the cup 78 within the prepared socket 90, in combination with a bone punch tool 18 deployed for optionally punching a shallow pilot hole in the patient bone 92 through one or more of the screw ports 86 formed in the cup 78 preferably in a superior position relative to the central port 82. This bone punch tool 18, or awl, is shown in more detail in FIG. 21 to comprise a short and pointed tip 98 in combination with a radially enlarged base or shoulder 100 which separates the sharp end tip 98 from the adjacent elongated tool handle 102. A region of the tool handle 102 near the sharp end tip 98 is bent as indicated in FIG. 21 by reference numeral 103 at an angle of about 30 degrees for facilitating tool manipulation and use. The entire bone punch tool 18 is constructed from a surgical grade metal, such as a sterilizable stainless steel or the like. In use, the pointed tip 98 is manually deployed to form as by punching a small pilot hole (about 15 mm in depth, and, when a 6.5 mm diameter bone screw 20 is used, a diametric size of about 3 mm) through the cortical bone overlying the more porous cancellous bone through one or more of the screw ports 86. In practice, if desired for additional fixation (beyond press-fitting) of the acetabular cup 78 relative to the prepared socket 90, one such pilot hole is typically formed to receive a self-tapping bone screw 20 (FIG. 22) which is rotatably installed into the pilot hole as by means of a rotary driver 106 or the like. In one preferred form, the self-tapping bone screw 20 has a diametric size of about 6.5 mm for self-tapping installation into the pilot port, and further into cancellous patient bone 92 (substantially without penetrating he opposite corticle bone). The specific screw length can vary according to patient anatomy.

FIG. 23 shows the bearing insert 84 seated within the concave side of the metal acetabular cup 78. In this regard, the bearing insert 84 has a generally hemispherical shape with an outer convex surface adapted to fit and seat closely within the concave side of the cup 78, in combination with a concave side of the bearing insert 84 sized shaped to rotationally receive and support the temporary ball-shaped cap 64 and/or the femoral ball 30 mounted respectively onto the neck portion 26′ of the femoral broach tool 16 (FIG. 12) or alternately mounted onto the neck portion 26 of the femoral component 22 (FIG. 1 5). The bearing insert 84 is formed from a high density and preferably molded and machinable plastic material, such as a high density polyethylene or the like. In use, the concave side of the bearing insert 84 is chosen to be about slightly greater than the diameter of the femoral ball 30 of the femoral prosthesis 12 (by about 0.5 millimeters (mm) maximum).

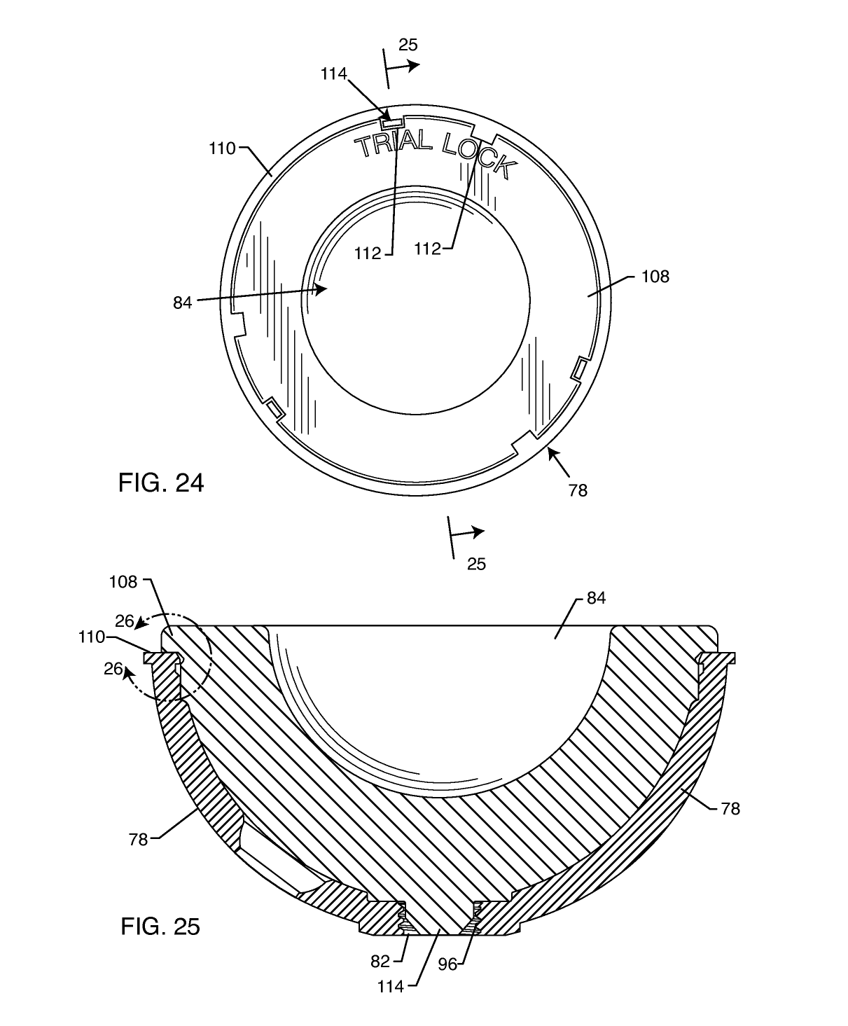

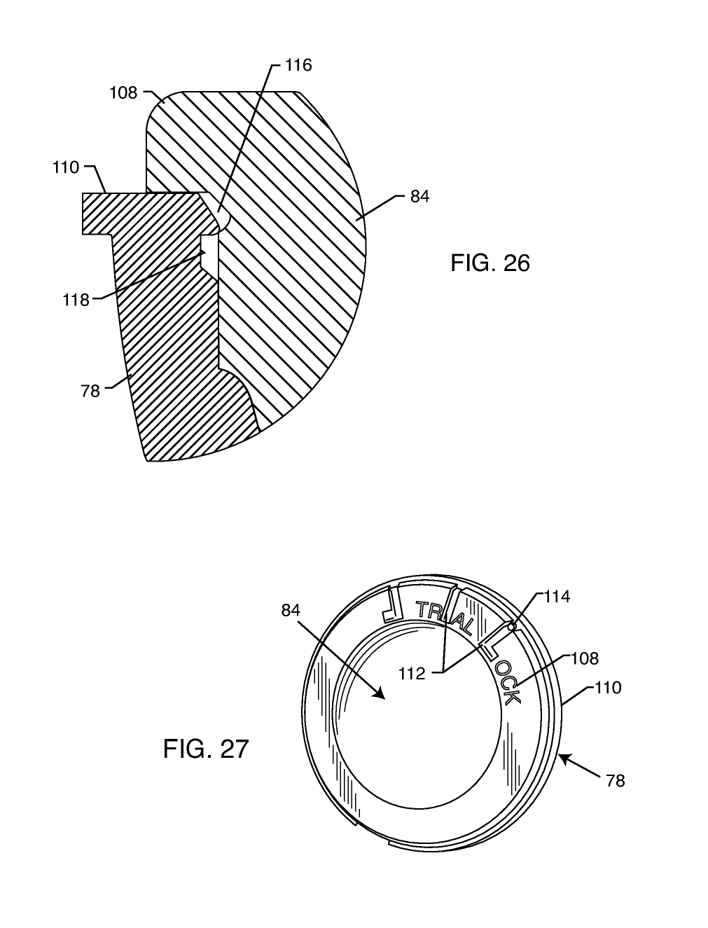

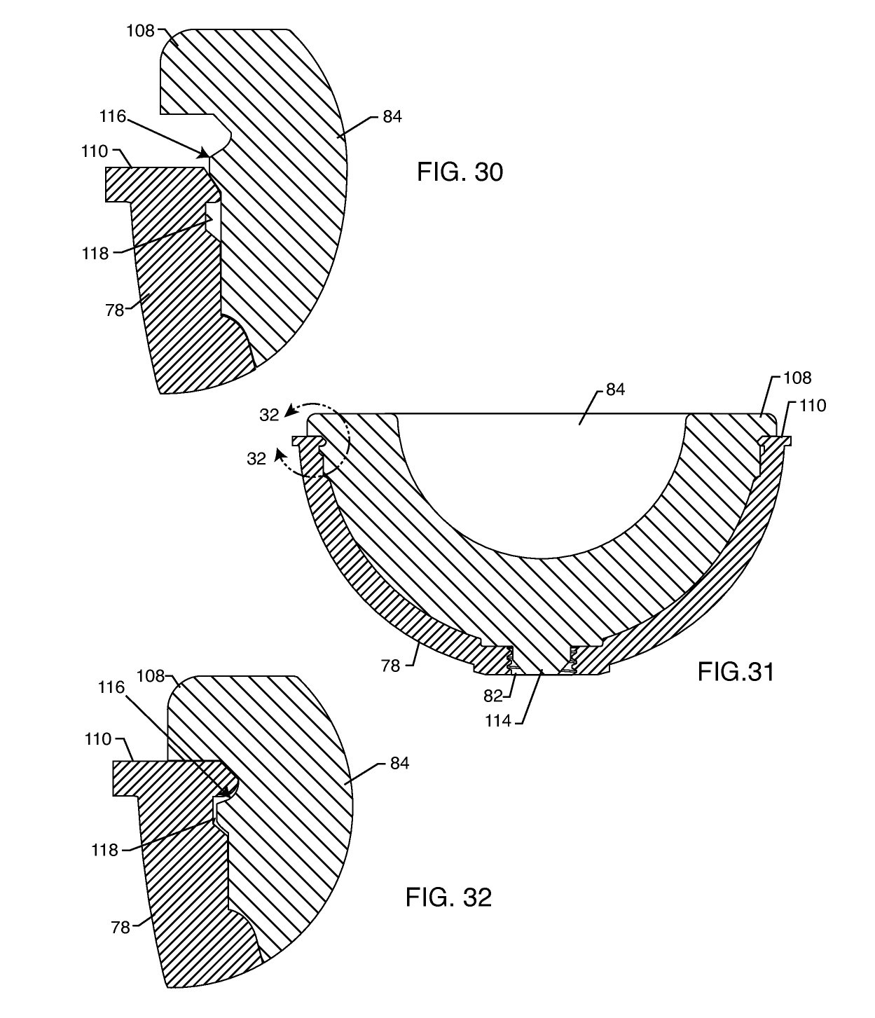

The bearing insert 84 includes a face flange 108 having a size and shape to slightly overlie an annular face ring 110 at the concave side of the acetabular cup 78. This face flange 108 has at least two notches 112 formed therein, respectively labeled “trial” and “lock” (shown best in FIG. 24). When the “trial” notch 112 is aligned with a short and radially in-turned tab 114 on the cup face ring 110 (again, shown best in FIG. 24), the bearing insert 84 is fully received and seated into the acetabular cup 78. FIG. 25 shows a central alignment post 114 on the bearing insert convex side seated fully into the central port 82, with the convex side of the bearing insert 84 seated and supported fully upon the concave side of the cup 78. In addition, the face flange 108 of the bearing insert 84 is seated fully and flush upon the face ring 110 of the acetabular cup 78 (FIGS. 25-26). In this position, detent surfaces 116 (FIGS. 29-30) on the bearing insert 84 are rotationally misaligned with mating detent surfaces 118 (FIGS. 25-26 and 29-30) formed within the cup 78 near the face ring 110, to accommodate full seating of the bearing insert without attachment to the cup 78.

In this “trial” position, the bearing insert 84 can thus be fully seated into the acetabular cup 78 for the purpose of a trial size and fit measurement with the temporary ball-shaped cap 64 (FIG. 12) mounted onto the neck portion 26′ of the femoral broach tool 16. During such trial fit measurement, the patient's leg is abducted and externally rotated to insure sufficient post-surgical clearance between the femoral and acetabular prostheses 12,14. If there is not, the implant components can be taken apart easily, and the position of the acetabular cup 78 adjusted within the prepared socket 90. If necessary, a different one of the screw ports 86 can be used to create a different pilot hole using the bone punch tool 18, following by subsequent reinstallation of a different self-tapping bone screw 20 through the different screw port 86 into the newly formed pilot hole. The trial size and fit measurement can then be made again—noting that the head of the bone screw 20 must not project upwardly beyond the concave surface of the acetabular cup 78. Importantly, the bearing insert 84 is freely removable from the cup 78 to permit cup positional adjustment, and is then freely re-installed into the cup 78 for the subsequent trial size and fit measurement. In other words, the bearing insert 84 is not destroyed by removal and/or re-insertion into the cup 78 in the “trial” position, whereby only one bearing insert 84 is required during surgery.

After the trial size and fit measurement has been satisfactorily concluded, the temporary ball-shaped cap 64 (FIG. 12) is removed from the femoral broach 16, and the inserter-retractor tool 58 (FIG. 10) is employed to retract the femoral broach 16 from the resected femur 42. Thereafter, the femoral component 22 is seated within the resected femur 42 (FIGS. 13-14) and the femoral ball 30 (FIG. 15) is attached onto the neck portion 26. Interposed tissue between the bearing insert face flange 108 and the underlying face ring 110 of the acetabular cup 78 must be avoided to insure full and proper seating of the bearing insert 84 within the cup 78.

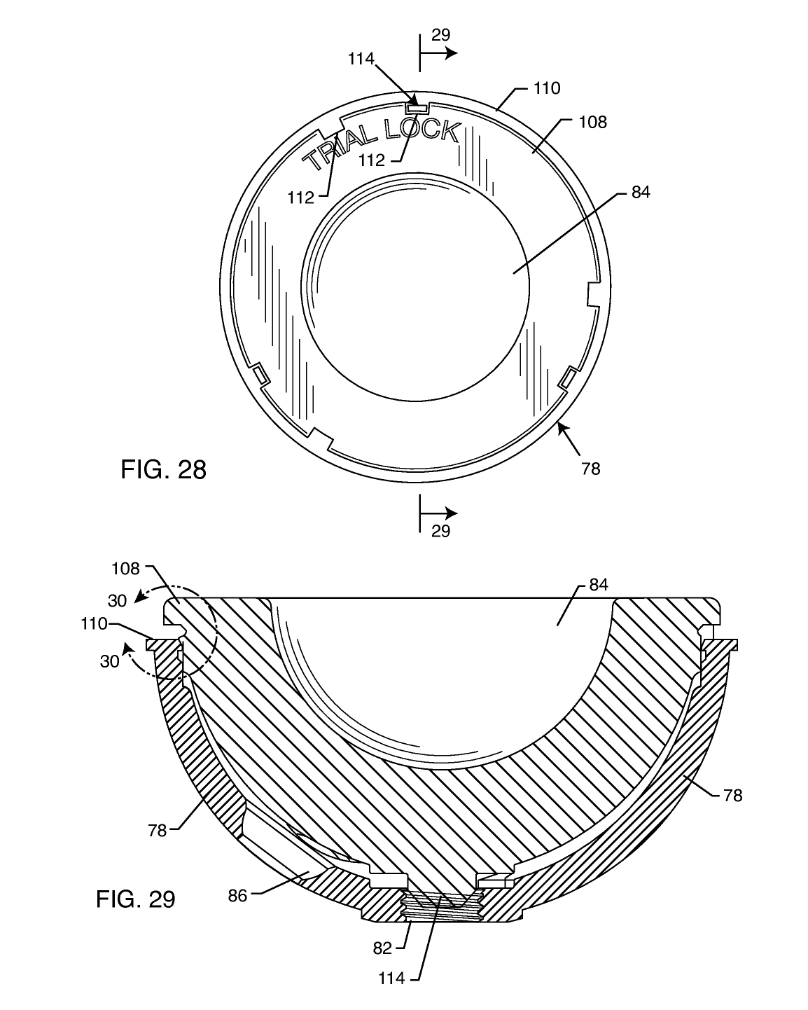

The bearing insert 84 is removed from the acetabular cup 78 and re-positioned relative thereto with the “lock” notch 112 aligned with the in-turned tab 114 on the cup 78 (FIGS. 27-28). In this position, as viewed best in FIG. 30, the detent surfaces 116 on the bearing insert 84 are rotationally aligned with the matingly shaped detent surfaces 118 on the interior of the acetabular cup 78, whereby the convex surface and the face flange 108 of the bearing insert 84 are spaced respectively a short distance from the associated concave surface of the cup 78 and the face ring 110 on the cup.

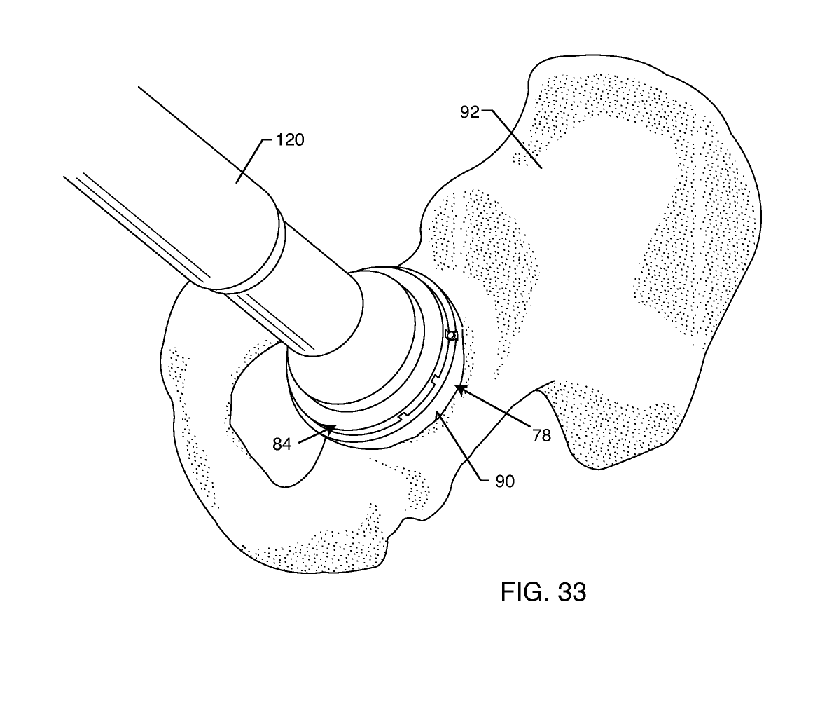

FIGS. 31-32 show the mating detent surfaces 116,118 fully engaged, with the bearing insert 84 fully seated and locked in position within the acetabular cup 78. Such full seating of the bearing insert 84 is achieved by use of an impact tool 120 shown in FIG. 33 and suitably driven by the mallet 48 (not shown in FIG. 33) for snap-fit engagement between these rotationally aligned detent surfaces 116, 118 in response to one or two sharp blows by the hammer 48 on the impact tool 120. In this regard, the acetabular cup 78 preferably includes a reduced wall thickness in the region of the detent surfaces 118 near the face ring 110 (FIGS. 29 and 32), to accommodate slight radial expansion (in combination with slight radial contraction of the bearing insert 84) as the impact tool 120 is manually impacted by the surgical mallet 48. FIG. 32 is an enlarged view showing full engagement of the bearing insert detent surface 116 in the form of a short radially extending tab into the cup detent surface 118 in the form of an undercut slot beneath a radially in-turned tab on the cup 78.

A variety of further modifications and improvements in and to the improved total hip arthroplasty of the present invention will be apparent to those persons skilled in the art. Accordingly, no limitation on the invention is intended by way of the foregoing description and accompanying drawings, except as set forth in the appended claims.

1-32. (canceled)

33. A bone punch tool for use in punching a bone screw pilot hole of predetermined size and depth into patient bone, said tool comprising:
an elongated tool handle;
a base at one end of said handle and defining a radially enlarged shoulder; and
an end tip projecting from said base and terminating in a sharpened point, said end tip being usable to punch said pilot hole into patient bone, said radially enlarged shoulder limiting the depth of said pilot hole.

34. A broach tool for shaping the medullary canal of a resected patient femur for secure seated implantation of a femoral component, said tool comprising:
a femoral tool component including external cutting teeth formed on the exterior surfaces of a distal end tip, a central region, and an upper end region thereof, said a femoral tool component having a medial-lateral taper that progressively increases from a distal end tip through said central region and at least over part of said upper region, said femoral tool component further having an anterior-posterior taper that progressively increases from said distal end tip through said central region and further through said upper region, and said femoral tool component further having a lateral-to-medial taper that progressively decreases from a lateral side to a medial side thereof.

35. The broach tool of claim 34 wherein said femoral tool component is formed from a selected biocompatible metal material.

36. The broach tool of claim 34 wherein said medial-lateral taper is about 5°.

37. The broach tool of claim 34 wherein said anterior-posterior taper is about 3-4°.

38. The broach tool of claim 34 wherein said lateral-to-medial taper is about 4-5°.

39. The broach tool of claim 34 wherein said medial-lateral taper is about 5°, said anterior-posterior taper is about 3-4°, and said lateral-to-medial taper is about 4-5°.

40. The broach tool of claim 34 wherein said femoral tool component further includes a line formed on the exterior thereof corresponding with a top edge of a porous bone ingrowth surface on a femoral component.

41. The broach tool of claim 34 wherein said femoral tool component further includes a neck region for supporting a ball-shaped cap.
